(19) United States
(12) Patent Application Publication
(10) Pub. No.: US 2026/0144437 A1
(43) Pub. Date: May 28, 2026

(54) Endoscope Device and Endoscope

(71) Applicant: KARL STORZ SE & Co. KG, Tuttlingen (DE)

(72) Inventor: Beat Krattiger, Beringen (CH)

(73) Assignee: KARL STORZ SE & Co. KG, Tuttlingen (DE)

(21) Appl. No.: 19/121,996

(22) PCT Filed: Oct. 24, 2023
(86) PCT No.: PCT/EP2023/079612

§ 371 Date: Apr. 17, 2025

(30) Foreign Application Priority Data
Oct. 25, 2022 (DE) 10 2022 128 272.4

(51) Int. Cl. A61B 1/12

(52) U.S. Cl. CPC A61B 1/128

(57) ABSTRACT

The invention relates to an endoscopic device comprising a shaft, wherein the shaft comprises: a distal shaft end and a proximal shaft end opposite the distal shaft end; an outer casing extending from the distal shaft end to the proximal shaft end; a shaft inner section which is at least partially surrounded peripherally by the outer casing and arranged between the distal and the proximal shaft ends; at least one inner casing extending at least partially in the shaft inner section and defining at least one function space; and a closed shaft intermediate space, arranged in the shaft inner section and outside the functional space, in which a heat transfer unit is arranged, which at least largely fills the shaft intermediate space and is designed to dissipate heat from the distal shaft end.

The invention relates to an endoscopic device and an endoscope comprising the endoscopic device.

The prior art has disclosed endoscopic devices with a shaft. The shaft is usually an elongate element, similar to a pipe, which is provided for insertion into a body-internal cavity of a human or animal body through a natural or an artificially created body orifice. The shaft usually has a rigid embodiment and is coupled or couplable to a handle or any other device for actuating the shaft. Conventionally, the shaft has a hollow embodiment in the interior, and so functional units may be guided through it.

A cross section through an endoscopic shaft according to the prior art is depicted in FIG. 2. By way of example, the functional units such as a working channel 12, a fluid channel 14, a light guide bundle 16, a current conductor bundle 18, an image guide bundle and/or a rod lens system may extend through the shaft 10. Moreover, the German patent application DE 10 2019 003 839 A1 has disclosed an endoscopic shaft through which two heat pipes (heat pipe 20) extend at least in part in order to dissipate heat from a head end of the shaft. The heat pipes are embedded in a drilled hole in the shaft pipe. Further, the heat pipes are thermally coupled to a handpiece of the endoscope by means of a heat-conducting adhesive such that the heat absorbed at the head end of the shaft can be dissipated to the handpiece. However, heat pipes also already develop their effect by heat emission via the surface along their longitudinal extent.

Heat pipes are known from the prior art. They are usually formed from a hermetically sealed copper pipe, the interior of which contains a capillary layer and a cavity for the exchange of gases. The enthalpy of vaporization of a working medium situated in the copper pipe, for example water, acetone or ammonia, is used for the heat transfer. Further, this may be a mixture of liquids and/or gases. In the case of a heat input above the boiling point of the working medium, the latter starts to evaporate, leading to a local pressure increase. Consequently, the vapor flows into regions of lower pressure. In these regions of lower pressure, the temperature of the working medium drops below the boiling point, whereby the working medium condenses and energy is output to the jacket of the heat pipe. This occurs especially in those regions where the heat pipe is cooled. The condensed working medium is then guided back through the capillary layer and by way of the effect of adhesion forces in the direction of the heated region of the heat pipe.

The disadvantage of the use of conventional heat pipes for dissipating heat in an endoscopic shaft is that the heat pipes require much space and thus increase the shaft diameter or reduce the working space for the feedthrough of functional units, for example. Moreover, the heat pipes increase the weight of the shaft, whereby the handling of the latter is made more difficult. Further, this additional weight must be carried by the shaft, in particular by the outer jacket thereof. In the process, the heat pipes usually made of copper pipes virtually do not contribute to the desired stability of the shaft due to the easy deformability of copper materials. Consequently, the shaft needs to be formed to be very stable for a desired rigid embodiment of the endoscopic shaft with heat pipes arranged therein, contributing to a further increase in weight.

However, modern endoscopic shafts require technical solutions for heat dissipation since the progressive miniaturization of imaging devices, of computer chips and of illumination units offers the possibility of arranging ever more waste heat-producing functional units at the head end (distal end) of the endoscopic shaft. For example, an LED camera may thus be arranged at the distal shaft end of the shaft. Much space is saved in this way, and the imaging quality is improved. At the same time, significant amounts of waste heat arise during the operation of the camera and further miniaturized functional units, for example LED lights, arranged distally.

Using the prior art as a starting point, the invention addresses the problem of providing a structurally efficient heat transport structure.

According to the invention, this problem is solved by an endoscopic device and an endoscope, as described herein and defined in the claims.

The features according to the invention provide a heat transport structure that is structurally efficient, especially in view of an effectiveness of the heat transfer, an exploitation of installation space available and a high degree of stability with a low weight at the same time. It is possible to provide an endoscopic device and an endoscope with effective heat emission. Moreover, it is possible to ensure a good operability of the endoscopic device in relation to a compact, rigid and light embodiment of the endoscopic device. It is possible to provide an efficient solution for cooling the shaft in order to avoid overheating in tissue regions during surgical procedures and an attendant injury to the tissue. Moreover, it is possible to meet the need for ever improving illumination quality, image sharpness and faithful color scheme by the use of miniaturized functional units at the distal shaft end.

The endoscopic device comprises a shaft which comprises a distal shaft end and a proximal shaft end opposite the distal shaft end. By preference, the shaft is a shaft that extends in the longitudinal direction, for example in the form of a pipe or a rod. By preference, the shaft has a rigid embodiment and may be bent.

The shaft also comprises an outer jacket that extends from the distal shaft end to the proximal shaft end and an internal shaft portion that is at least partially enclosed circumferentially by the outer jacket and is arranged between the distal and the proximal shaft end. The internal shaft portion may be sealed at its longitudinal ends. For example, the internal shaft portion may be a sealed or hermetically sealed portion.

Further, the shaft comprises at least one inner jacket that extends at least partially in the internal shaft portion and that defines at least one functional space. In an embodiment, the inner jacket or/and the functional space may extend at least partially through the internal shaft portion from the distal shaft end to the proximal shaft end. Hence the functional space may also be referred to as a feedthrough channel. Hence, an endoscopic functional unit may be guidable from the distal shaft end to the proximal shaft end through the functional space in the form of a feedthrough channel. For example, the endoscopic functional unit may be a working channel pipe, a fluid channel pipe, a light guide bundle, a current conductor bundle or a conventional heat pipe. Alternatively, the functional space may also be configured as a rinsing channel or/and aspiration channel for supplying or/and removing fluids. The functional space may also be a feedthrough channel in this case. For example, the at least one functional space may be rod-shaped or tubular or/and may be bent in accordance with the shape of the shaft. The functional space may receive one or more endoscopic functional units.

In some embodiments, the inner jacket or/and functional space may extend partially into the shaft, for example extend into the latter at a proximal end. For example, this is suitable for receiving an endoscopic functional unit that is configured for wireless data transfer, for example. Exemplary functional units include an illumination device, a camera or/and a camera chip. The endoscopic functional unit may be arranged at the distal shaft end. The functional space may have any desired shape, for example have the shape of a blind hole or be a quadrilateral or half-round cutout.

In some embodiments, at least one functional space may take the form of a feedthrough channel and at least one further functional space may extend into the shaft without passing fully through the latter.

Moreover, the shaft comprises a sealed shaft interspace which is arranged in the internal shaft portion and outside of the functional space and in which a heat transfer unit is arranged, the latter filling at least a majority of the shaft interspace and being configured to dissipate heat from the distal shaft end. In this context, the expression “at least a majority” should be understood to mean in particular at least to an extent of 55%, by preference at least to an extent of 65%, preferably at least to an extent of 75%, particularly preferably at least to an extent of 85% and very particularly preferably at least to an extent of 95%, and advantageously all, to be precise in particular in relation to a volume and/or a mass of an object. The shaft interspace may be hermetically sealed in an embodiment.

The sealed shaft interspace with the heat transfer unit arranged therein may form a heat pipe. A heat pipe is distinguished in that the latter enables efficient energy transport, i.e. heat transport. It is understood that the heat pipe formed is based on the same physical laws as conventional heat pipes. However, the difference may consist in the fact that the heat pipe formed is integrated into the endoscopic shaft and consequently contributes to the saving of installation space, saving of costs and saving of weight in relation to the endoscopic device.

The heat transfer unit may be arranged in the internal shaft portion but outside of the at least one functional space. In this way, the heat transfer unit may be integrated into the shaft in space-saving fashion. Moreover, it is possible to form the heat transfer unit without a copper pipe, whereby an increase in weight of the shaft is avoided.

In some embodiments, the heat transfer unit is directly and/or immediately in contact, especially in extensive contact, with the outer jacket or/and the inner jacket. Alternatively, the heat transfer unit may be formed at least partly integrally with the outer jacket or/and the inner jacket. Within the scope of this disclosure, “integrally” may mean in one piece and/or in one part and/or directly connected to one another.

The heat transfer unit or the shaft interspace is preferably hermetically sealed at the distal and proximal side by means of a closure. Consequently, a sealed shaft interspace, preferably a hermetically sealed shaft interspace, may be formed between the outer jacket, the inner jacket and the proximal and distal closure of the heat transfer unit.

Opposite distal and proximal shaft ends may be thermally coupled to one another by means of the heat transfer unit or the heat pipe formed, whereby, for example, heat can be dissipated from the distal shaft end to the proximal shaft end. Further, heat can be emitted along the longitudinal extent of the shaft by the outer jacket of the shaft by means of the heat transfer unit or the heat pipe formed.

In some embodiments, the heat transfer unit comprises a gas exchange volume and a fluid transport layer. The fluid transport layer may comprise at least one porous element and/or be designed as such, for example a capillary layer. The entire shaft interspace may be used as gas exchange volume and at least a portion of the surfaces delimiting the shaft interspace, in particular at least a majority of those surfaces, may be designed as fluid transport layer. Expressed differently, the gas exchange volume and the fluid transport layer may be arranged in the shaft interspace. In this way, the shaft interspace available in the shaft in any case is used effectively for the heat transport, and the provision of the heat transfer unit requires no additional space in the shaft.

In some embodiments, the fluid transport layer is arranged on an inner face of the outer jacket or/and arranged on an outer face of the inner jacket. Consequently, the fluid transport layer may be situated in the shaft interspace and may protrude into the latter. By preference, the fluid transport layer and the outer jacket or the inner jacket are formed integrally. At least one portion of the inner face of the outer jacket or/and at least one portion of the outer face of the inner jacket may be covered by the fluid transport layer. In this way, lateral surfaces in the shaft interspace that are available in any case are used effectively for the formation of the heat transfer unit. The outer jacket or/and the inner jacket thus adopts/adopt a dual function in particular: the shaft is provided with stability and moreover capillary fluid transport for the heat transfer unit is formed.

Further, such an embodiment of the heat transfer unit may contribute to saving weight in comparison with the additional weight when use is made of conventional heat pipes, the copper outer jacket of which provides no additional stability for the shaft but instead even loads the latter with additional weight and consequently reduces stability. In relation to the choice of material, this means that copper pipes conventionally used in heat pipes make little contribution to the stability of the shaft as they are easily deformable. If no additional copper pipes are required for the heat pipe since the heat pipe is integrated into the shaft or its outer jacket, then the weight of the copper pipes is dispensed with. The shaft becomes more stable. Moreover, if the inner jacket is manufactured from steel, in particular stainless steel, and also used to form the heat pipe, then the effectiveness of the heat transport is increased. Moreover, further steel pipes within the shaft additionally contribute to the stability.

It may furthermore be advantageous for the fluid transport layer to be arranged directly and/or immediately on an inner face of the inner jacket. Moreover, an intermediate layer, for example an adhesive layer, may be formed at least in portions between the outer jacket or/and the inner jacket and the fluid transport layer. The intermediate layer may improve the heat transfer at the respective jacket on the one hand and contribute on the other hand to a better bond between the fluid transport layer and the respective jacket.

In an alternative to that or in addition, the fluid transport layer may, at least in portions, be applied directly to the outer jacket or inner jacket, e.g. by grooving, cold forming, stamping, machining, grinding or other surface-increasing methods. The fluid transport layer may further be formed or optimized by chemical modification and/or topographic structuring of the surface.

The fluid transport layer may comprise a wick structure and/or comprise grooves. The fluid transport layer may be constructed, at least in portions, as a spongy structure, for example produced by means of a sintering method. The fluid transport layer may comprise a fabric structure or a fiber structure.

Further, the heat transfer unit may comprise at least one heat transport means. The heat transport means may be based on gas exchange, liquid transport and enthalpy of vaporization of the heat transport means. The evaporable heat transport means may be present in freely movable fashion in the gas exchange volume and the fluid transport layer of the heat transfer unit. The amount of evaporable heat transport means is determined by the overall volume of the heat transfer unit and the dimension of the fluid transport layer. The heat transport means may be present in the fluid layer and in the gas exchange volume. For example, the heat transport means comprises water, acetone or ammonia. Further, this may be a mixture of liquids and/or gases.

For an installation space-efficient arrangement, at least 90%, by preference at least 95%, of a cross-sectional area of the shaft interspace between the outer jacket and the at least one inner jacket may be filled with the fluid transport layer and the heat transport means.

In some embodiments, the endoscopic device may further comprise at least one of a proximal and a distal thermal connection piece for heat emission and heat absorption, respectively. The heat emission, for example in the form of thermal energy, may be implemented from the heat transfer unit to a heat sink that is thermally coupled to the thermal connection piece. For example, the heat transfer unit may absorb heat from a waste heat-creating functional unit that is thermally coupled to the connection piece, e.g. from a CMOS chip, an illumination unit, an image recording device, a radio transmitter, a signal amplifier or a light transmitter/receiver for a fiber-optic data transfer. The thermal connection piece improves a local heat distribution and heat transfer between the heat transfer unit and a further element adjoining the latter. A heat transfer may be obtained by the use of a thermal connection piece. The thermal connection piece may protrude beyond the internal shaft portion or/and extend into the shaft interspace. The shaft interspace may be sealed, preferably hermetically sealed, by means of the thermal connection piece. The thermal connection piece may be the distal or/and proximal closure of the heat transfer unit or of the shaft interspace.

A further improvement in the heat transfer may be obtained by virtue of the at least one thermal connection piece comprising a surface-increasing means for improving the heat emission or heat absorption, wherein the surface-increasing means is formed within the sealed region of the heat transfer unit and projects into the latter or/and is formed outside of the sealed region of the heat transfer unit and points away from the sealed space of the heat transfer unit.

The term surface-increasing means may include all conceivable surface-increasing structures, for example blind holes, rods, pipes, wires, plate/sheet, at least one rolled-up PGS thermal conduction film, a metallized unit, a metallized PGS thermal conduction film, e.g. galvanically copper-plated, provided with a graphene layer or provided with a pyrolytic graphite film. Furthermore, a capillary layer at the surface represents a surface-increasing means.

The surface-increasing means may comprise a fluid transport layer and/or may be arranged in the direct vicinity of same. For example, a rod or/and a plate may be provided with a capillary layer, wherein the capillary layer increases the surface of the rod or/and the plate.

A further improvement in the heat transfer may be obtained by virtue of the surface-increasing means being produced from a thermally conductive material. Suitable thermally conductive materials comprise copper, copper alloys, silver, gold, aluminum, graphite or graphene.

In addition to that or in an alternative, the heat transfer may be improved by virtue of the outer jacket or/and the inner jacket comprising a thermally conductive element, in particular a thermally conductive layer, the thermal conductivity of which is higher than that of the material of the outer jacket or of the inner jacket. For example, an inner wall of the outer jacket or/and an outer wall of the inner jacket may be copper-plated.

The outer jacket or/and the inner jacket may be produced from stainless steel, e.g. high-grade steel, medical-grade steel, autoclavable steel. High-grade steel is advantageous in that it is resistant to corrosion and mechanically loadable, i.e. hard and stable.

In some embodiments, the endoscopic device further comprises a functional unit that is arranged and/or arrangeable at the distal shaft end, creates waste heat and is thermally coupled or thermally couplable to the heat transfer unit such that heat from the functional unit that creates waste heat can be supplied via the distal thermal connection piece to the heat transfer unit.

In an embodiment, the distal thermal connection piece may comprise the functional unit that creates waste heat. Alternatively, the distal thermal connection piece may be in indirect thermal contact with the functional unit that creates waste heat, via a solid body or/and via an outer heat transport fluid.

For example, the functional unit that creates waste heat comprises an electrical functional unit. By preference, the electrical functional unit comprises at least one CMOS chip, at least one illumination unit, at least one effector unit or/and at least one camera unit. The effector unit may comprise a mill, laser, RF applicator, US applicator, thermal applicator, a medicament unit, a scalpel, forceps, scissors, etc. To realize a stereo recording for example at the distal shaft end, the electrical functional unit may comprise a plurality of CMOS chips. The illumination unit may comprise a plurality of LEDs that differ in terms of their wavelength in order to illuminate different aspects and set the light heat. The effector unit may be a laser unit for laser cutting of tissue. The electrical functional unit may be configured to enable multispectral imaging and may for example comprise a multispectral camera to this end. For example, a plurality of LEDs may be used to provide a multispectral mode, a fluorescence mode, a white-light mode or/and a hyperspectral mode.

The functional unit that creates waste heat may be controlled and/or controllable by means of a wireless communications technology or/and be controlled and/or controllable by means of a wired connection. The at least one functional space may be configured to receive at least one functional unit that protrudes at least partially into the internal shaft portion or passes through the internal shaft portion. In an embodiment, the functional space may further receive the wired connection between a controller of the functional unit and the functional unit. The functional unit may distally or/and proximally protrude beyond the sealed shaft interspace. Examples of a functional unit that creates waste heat comprise a light guide bundle, a current conductor bundle, a surgical tool or/and a camera. The functional unit may be the functional unit that creates waste heat or any other desired functional unit.

In some embodiments, the internal shaft portion may receive a conventional heat pipe that is surrounded by a copper pipe. In that case, the outer jacket of the copper pipe of the heat pipe may comprise a heat pipe-copper outer jacket, which is adjoined by the shaft interspace. In this case, the heat pipe-copper outer jacket may be equipped with a further fluid transport layer, for example a capillary layer, for fluid transport. The cross section of the endoscopic shaft is exploited effectively in such an embodiment.

The use of an additional conventional heat pipe arranged in the internal shaft portion allows for the use of two different heat transport fluids which for example differ in terms of their behavior, i.e. whose vapor pressure curves, temperature ranges, viscosities, enthalpies of vaporization or influences of gravity on the capillary fluid line may differ. This may be advantageous when maximizing the heat dissipation since the different heat transport fluids can be better adapted to different conditions. Further, the breakdown of the heat transport in the case of a thermal overload might be implemented continuously or in stages, and so the breakdown of the heat transport may be recognized in a timely manner and hence the amount of waste heat to be dissipated may be curbed in a timely manner in order to counteract damage due to overheating.

By preference, all functional units that should be guided through the internal shaft portion and/or should be received therein are arranged in the at least one functional space and sealed, preferably hermetically sealed, vis-á-vis the shaft interspace by means of the inner jacket. The functional units may comprise the functional unit that creates waste heat or any other desired functional unit.

The endoscopic device may further comprise a handle that is coupled or couplable to the proximal shaft end or a region adjacent to the proximal shaft end. For example, the handle is a handpiece that is functionally couplable to the endoscopic device. The handle may comprise actuation elements in order to actuate the functional units received in the internal shaft portion.

In a preferred embodiment, the endoscopic device or at least its shaft is autoclavable.

According to a further aspect, the invention comprises an endoscope that comprises an endoscopic device as claimed in any of the preceding claims. Therefore, the embodiments and advantages presented above in relation to the endoscopic device apply equally to the endoscope.

An endoscope according to the invention may serve to dissipate heat from instruments, for example a scalpel, forceps, scissors, etc., in electrosurgery. This can prevent tissue treated by the endoscope from burning or adhering to the endoscope, in particular to the proximal shaft end of the shaft.

The present invention is described below by way of example and with reference to the accompanying figures. The drawing, the description and the claims contain numerous features in combination. A person skilled in the art will advantageously also consider the features individually and use them in meaningful combinations within the scope of the claims.

If there is more than one instance of a given object, only one of them may be referenced in the figures and in the description. The description of this instance can be transferred accordingly to the other instances of the object. If objects are named in particular by means of numerical words, such as first, second, third object, etc., these are used to name and/or assign objects. Thus, for example, a first object and a third object might be included, but no second object. However, a number and/or a sequence of objects could additionally also be derivable from numbers in that case.

### In the drawing:

FIG. 1 shows a schematic illustration of an endoscope comprising an endoscopic device, an imaging device and an illumination device;

FIG. 2 shows a schematic cross-sectional illustration of an endoscopic shaft according to the prior art;

FIG. 3 shows a schematic cross-sectional illustration of an embodiment of an endoscopic device according to the invention;

FIG. 4 shows a schematic cross-sectional illustration of an embodiment of an endoscopic device according to the invention;

FIGS. 5a-5b show a schematic illustration of a longitudinal section through an embodiment of an endoscopic device according to the invention;

FIGS. 6a-6b show a schematic illustration of a longitudinal section through an embodiment of an endoscopic device according to the invention; and

FIG. 7 shows a schematic illustration of a further endoscopic device.

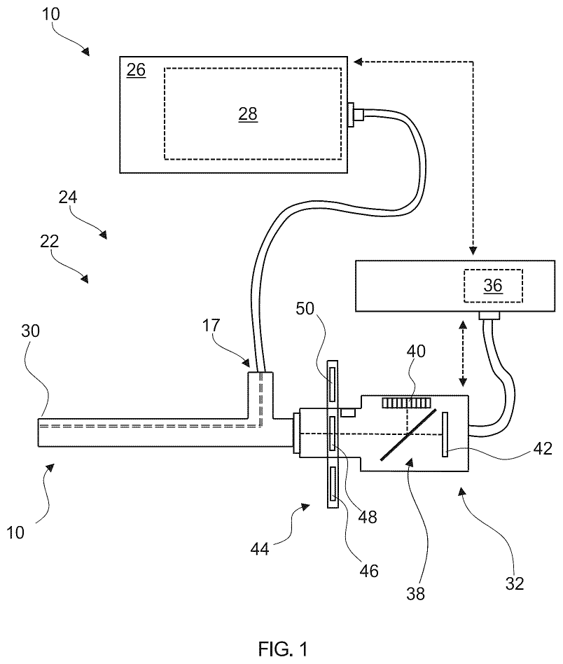

FIG. 1 shows a schematic illustration of an endoscope 22 having an endoscopic imaging device 24. For example, the imaging device 24 is provided for examining a cavity. Furthermore, the imaging device 24 comprises an illumination device 26 and an illumination unit 28. The illumination unit 28′is configured to supply illumination light to an optical interface 17. In an alternative to that or in addition, an illumination device 26 or/and the illumination unit 28 may be arranged at a distal shaft end 30, as will be explained in more detail below. This arrangement is preferably usable when the illumination device 26 or/and the illumination unit 28 can be embodied to be very small, for example when miniaturized LED light sources are used.

In the depicted case, the endoscope 22 also comprises a display unit on which images can be displayed, the images being based on image data acquired by means of the imaging device 24. These may be video images, freeze frames, overlays of different images, partial images, image sequences, etc.

Further, the endoscope 22 comprises a camera unit 32′ and a shaft 10. In FIG. 1, the camera unit 32 is coupled or couplable to a proximal shaft end 34, wherein the shaft 10 is optically coupled to the camera unit 32. In an alternative to that or in addition, a camera unit 32 may be arranged at the distal shaft end 30. This arrangement is preferably usable when the camera unit 32 has been miniaturized to such an extent that it is insertable into the shaft 10. The camera unit 32 is connected to a controller 36 in a wireless or wired manner.

The camera unit 32′ comprises imaging sensor system 38′, by way of example a white-light sensor 40′and a near IR sensor 42′ in the present case. Expressed in general terms, the imaging sensor system 38′ may comprise one or more light sensors/image sensors with at least spatial resolution, for example at least one CMOS sensor and/or at least one CCD sensor.

The endoscope 22 comprises a filter unit 44′ with optical filters 46′, 48′, 50′. Three optical filters have been depicted by way of example; however, it is understood that a different number may be used. The filter unit 44′ is switchable between a multispectral mode and a fluorescence mode. Furthermore, the filter unit 44′ may additionally be switchable into a white-light mode and/or into a hyperspectral mode. In an alternative to that or in addition, a filter unit 44 may be arranged at the distal shaft end 30. This arrangement is preferably usable when the filter unit 44 has been miniaturized to such an extent that it is insertable into the shaft 10. The filter unit 44 is connected to a controller 36 in a wireless or wired manner.

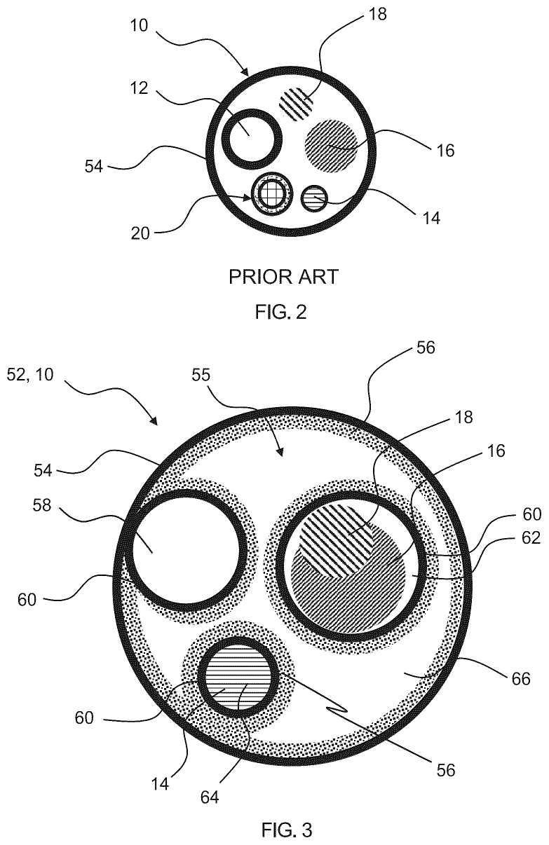

The shaft 10 comprises a plurality of elements, for example as explained in the introductory part relating to the prior art and as depicted in the cross section of FIG. 2. One or more of a working channel, a fluid channel 14, light guide bundle 16, a current conductor bundle 18, image guide bundle and/or a rod lens system and a heat pipe 20 are arranged in the shaft 10. Functional elements, for example a surgical instrument or/and an illumination device 26 or/and a camera unit 32, may be guided from the proximal shaft end 34 to the distal shaft end 30 by means of the working channel 12 or may be securely installed in the distal end 30. The fluid channel 14 is configured for a fluid to flow through it and may consequently operate as a rinsing channel and/or aspiration channel. The light guide bundle 16 is configured to guide light that was emitted by the illumination device 26′ through the shaft 10. The current conductor bundle 18 is configured to guide electrical current through the shaft 10.

The conventional heat pipe 20 is configured to dissipate heat from the distal shaft end 30 to the proximal shaft end 34.

An embodiment of an endoscopic device 52 according to the invention is depicted in FIG. 3. The endoscopic device 52 comprises a shaft 10 which is depicted in FIG. 3 in cross section perpendicular to its longitudinal extent. The shaft 10 comprises an outer jacket 54. The outer jacket 54 at least partially surrounds an internal shaft portion 55. A fluid transport layer 56 is arranged on an inner face of the outer jacket 54.

Furthermore, the shaft 10 comprises a functional space 58 which for example may fulfill the function of the above-described working channel 12 and which may be configured as a feedthrough channel for feeding through surgical instruments. The functional space 58 is surrounded circumferentially by an inner jacket 60. The fluid transport layer 56 is also arranged on an outer face of the inner jacket 60. In the depicted exemplary embodiment, the fluid transport layer 56 lies directly against the outer jacket 54 or/and the inner jacket 60.

Further, in the embodiment depicted in FIG. 3, the shaft 10 comprises a second functional space 62 for receiving the light guide bundle 16 or/and current conductor bundle 18 and a third functional space 64 for forming a fluid channel 14. In the present case, the functional spaces 58, 62, 64 take the form of channels. It is understood that the shaft 10 may comprise any desired number of functional spaces 58, 62, 64 and is not limited to the exemplary illustration of exactly three functional spaces 58, 62, 64.

The at least one functional space 58, 62, 64 arranged in the shaft 10 is circumferentially surrounded by the inner jacket 60. In this way, a shaft interspace 66 is formed within the outer jacket 54 and outside of the inner jacket 60. The fluid transport layer 56 extends into the shaft interspace 66. The totality of the lateral faces 54, 60 surrounding the shaft interspace 66, or at least a portion thereof, is covered by the fluid transport layer 56.

The shaft interspace 66 is sealed, by preference hermetically sealed, and is arranged in the internal shaft portion 55 and outside of the at least one functional space 58, 62, 64. A heat transfer unit is arranged in the shaft interspace 66 and fills a majority of the shaft interspace 66, i.e. more than 90%, preferably more than 95%, of the shaft interspace 66 is used as heat transfer unit. The sealed shaft interspace 66 with the heat transfer unit arranged therein by preference forms a heat pipe for dissipating heat along the shaft 10. This formed heat pipe differs from a conventional heat pipe 20 in that it is integrated into the shaft 10 and constituent parts of the shaft 10, for example the outer jacket 60 of the latter, surround at least a majority of the heat pipe.

The heat transfer unit by preference comprises a gas exchange volume 67, the fluid transport layer 56 and at least one heat transport means that is based in particular on gas exchange, liquid transport and enthalpy of vaporization of the heat transport means. The embodiment of the shaft interspace 66 as a sealed space, preferably as a hermetically sealed space, allows the pressure therein to be reduced vis-á-vis the ambient pressure, i.e. allows a vacuum to be generated. By preference, all elements arranged in the shaft interspace 66 or in the heat transfer unit are designed such that these are insensitive to the heat transport means or/and low pressures, e.g. a vacuum. Units that are not insensitive to the heat transport means or/and low pressures are arranged in protected fashion within the inner jacket 60 of the at least one functional space 58, 62, 64.

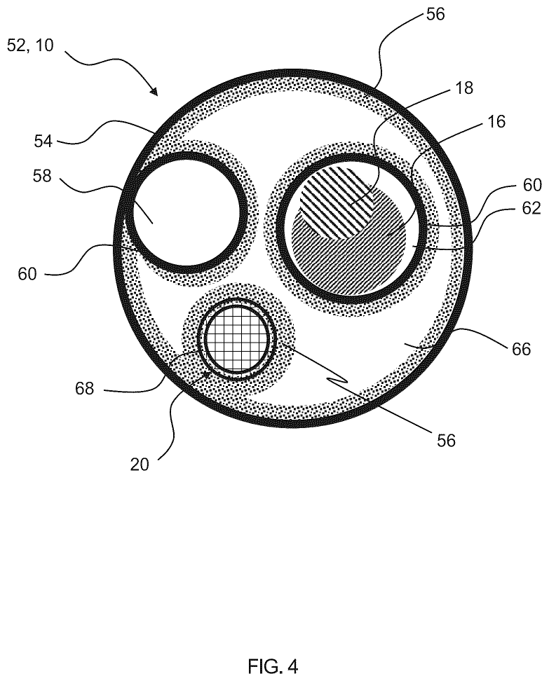

FIG. 4 shows a further embodiment of an endoscopic device 52 according to the invention. The endoscopic device 52 comprises the shaft 10 which is depicted in FIG. 4 in a cross section perpendicular to its longitudinal extent. The endoscopic device 52 depicted in FIG. 4 substantially corresponds to the endoscopic device 52 explained above in relation to FIG. 3. Therefore, only differences between the two embodiments according to FIGS. 3 and 4 are discussed below.

The shaft 10 depicted in FIG. 4 comprises a conventional heat pipe 20, which is sealed by a copper jacket 68. The conventional heat pipe 20 is arranged within the outer jacket 54, and the shaft interspace 66 extends between the copper jacket 68 and the outer jacket 54. The fluid transport layer 56 is arranged on an outer face of the copper jacket 68. Consequently, the conventional heat pipe 20 is integrated into the endoscopic device 52. The outer face of the conventional heat pipe 20 is used as boundary for the shaft interspace 66, in which the heat transfer unit is arranged. Consequently, heat is dissipated along the shaft 10 in two different ways, as a result of which there is a contribution to effective cooling.

It is understood that the endoscopic device 52 may comprise one or more functional spaces 58, 62, 64 and one or more conventional heat pipes 20.

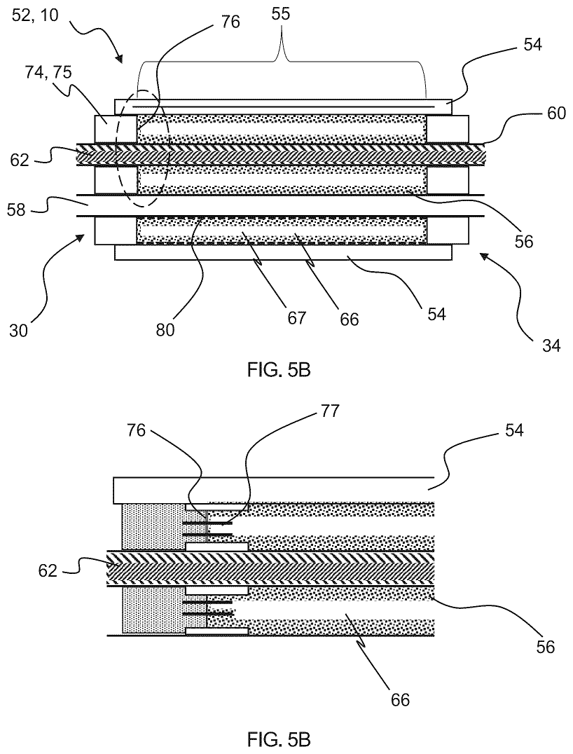

FIG. 5a shows a longitudinal section of the endoscopic device 52 in the longitudinal direction of the shaft 10 from a distal shaft end 30 to a proximal shaft end 34 opposite the distal shaft end 30. The internal shaft portion 55, which is at least partially surrounded by the outer jacket 54, is arranged between the distal shaft end 30 and the proximal shaft end 34. The sealed shaft interspace 66, in which the heat transfer unit is arranged, is provided in the internal shaft portion 55 and outside of the functional space 58, 62, 64. In the illustrated embodiment, the shaft interspace 66 is sealed at its longitudinal ends by way of a respective closure 74. The closure 74 may be in contact with an electrical functional unit 75 or may be arranged directly adjacently to the latter. Alternatively, the electrical functional unit 75 itself may form the closure 74 of the shaft interspace 66. In a preferred embodiment, the closure 74 and the electrical functional unit 75, or the shaft interspace 66 and the electrical functional unit 75, are in thermal contact.

The inner jacket 60 extends from the distal shaft end 30 to the proximal shaft end 34 or even beyond the latter. The inner jacket 60 passes through the closure 74 or the internal shaft portion 55 such that the shaft interspace 55 is sealed even if the inner jacket 60 passes through the latter. The inner jacket 60 may extend proximally or/and distally beyond the outer jacket 54.

FIG. 5b shows a detail of FIG. 5a, in which a thermal connection piece 76 for heat emission or heat absorption is arranged at the distal shaft end 30. However, the thermal connection piece 76 may also be arranged at the proximal shaft end 34 in addition to that or in an alternative. The thermal connection piece 76 may form the closure 74 and seal the shaft interspace 66. The inner jacket 60 may pass through the thermal connection piece 76. The thermal connection piece 76 may seal, preferably hermetically seal, the shaft interspace 66. In the depicted embodiment, the thermal connection piece 76 comprises a surface-increasing means 77 in the form of a plate or a pipe. Two surface-increasing means 77 are depicted by way of example; however, more or fewer surface-increasing means 77 may be used. The thermal connection piece 76 protrudes into the sealed shaft interspace 66 and moreover protrudes out of the sealed shaft interspace 66. In the depicted embodiment, the thermal connection piece 76 protrudes into the closure 74, i.e. ensures an effective heat transfer between the shaft interspace 66 or the heat transfer unit and the closure 74 adjacent thereto. It is understood that the thermal connection piece 76 may also be in thermal contact with any desired element, for example a functional unit 75 that creates waste heat.

The thermal connection piece 76 may be produced from at least one of copper, copper alloys, silver and aluminum. The surface-increasing means 77 may be produced from the same materials. For example, structuring on the surface of the thermal connection piece 76 represents the surface-increasing means.

Further, the thermal connection piece 76 may be produced with an offset drilled hole on the outside and twisted face on the inside for receiving thermally conducting films, sheets or tubules that have better thermal conductivity than steel pipes. This increases the thermally effective surface. As a result of the offset shaping of the thermal connection piece 76, steel pipes can be soldered in hermetically sealed fashion, and a thermal conduction film or a thermal conduction sheet can be joined, for example simultaneously or subsequently.

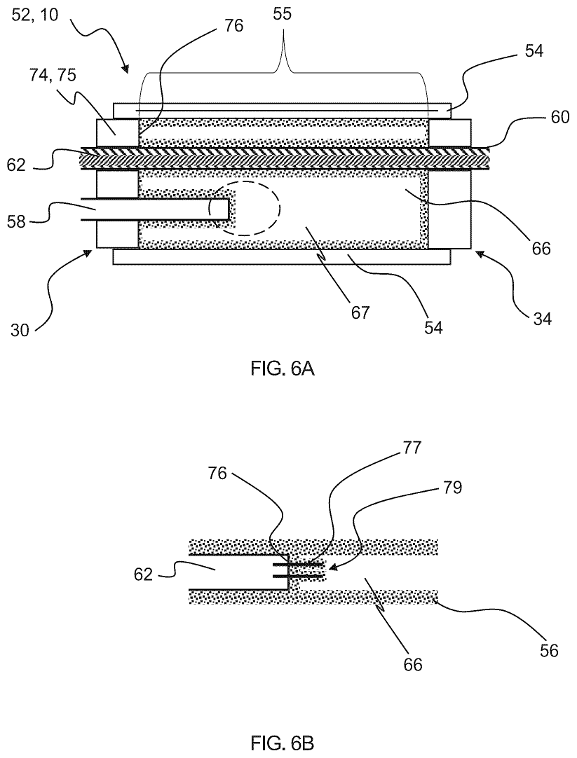

FIGS. 6a and 6b show a further embodiment of a shaft 10 according to the invention, in a longitudinal section along its longitudinal extent. The shaft 10 depicted in FIGS. 6a and 6b substantially corresponds to the shaft 10 that was explained above in relation to FIGS. 5a and 5b. Therefore, only differences between the two embodiments according to FIGS. 5a, 5b and 6a, 6b are discussed below.

Two different variants of the functional space 58, 62 are depicted in the longitudinal section through the shaft 10 depicted in FIG. 6a. The functional space 62 depicted in FIG. 6a corresponds to the functional space 62 depicted in FIG. 5a, and the functional space 58 is depicted in a further embodiment in which the functional space 58 only passes through one of the ends, preferably the proximal end, of the shaft interspace 66. For example, this embodiment allows the electrical functional unit 75 to be arranged in the shaft 10 adjacent to the distal shaft end 30. For example, the electrical functional unit 75 may communicate wirelessly with the controller 36. An exemplary electrical functional unit 75 to this end is a miniaturized illumination unit 18 or a miniaturized camera unit 32.

FIG. 6b shows a thermal connection piece 76 with a surface-increasing means 77 which in the depicted case moreover comprises a capillary layer 79, for example the fluid transport layer 56, on one surface in order to improve the heat transport to the thermal connection piece 76. The capillary layer 79 serves to increase the surface. Moreover, the surface-increasing means 77 in the depicted case has been formed by way of example with a plurality of ribs, which likewise serve to increase the surface.

In further embodiments, the heat transfer via the outer jacket 54 may be improved by virtue of the outer jacket 54 comprising a thermally conductive element 78, in particular a thermally conductive layer, the thermal conductivity of which is higher than that of the material of the outer jacket 54. A thermally conductive element 78 is depicted by way of example in FIGS. 5a and 6a. Further, the inner jacket 60 may comprise a thermally conductive element, the thermal conductivity of which is higher than that of the material of the inner jacket 60.

In all embodiments, the fluid transport layer 56 may comprise a capillary layer that directly or indirectly adjoins the inner face of the outer jacket 54 or the outer face of the inner jacket 60. The capillary layer may consist of sintered particles or of braided, woven, knitted, twisted or felted filaments. The capillary layer may be applied directly to the respective lateral face 54, 60, for example by means of a sintering method, or may be introduced into the latter, for example etched into the latter.

Alternatively, an intermediate layer 80 that is situated between the fluid transport layer 56 and the outer jacket 54 or the inner jacket 60 may be provided for connecting the fluid transport layer 56 to the respective jacket 54, 60. An exemplary intermediate layer 80 is depicted in FIG. 5a.

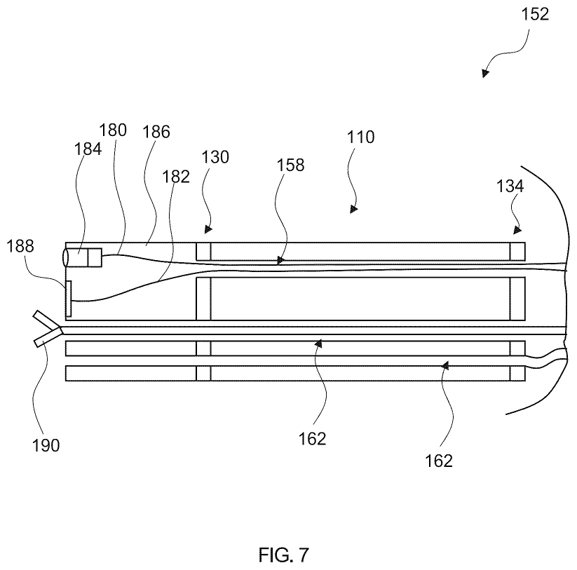

FIG. 7 shows a further embodiment of an endoscopic device 152 having a shaft 110. The shaft 110 also forms a heat transfer unit. In principle, the endoscopic device 152 may be constructed as described above. Differences in the configuration are predominantly discussed below.

The shaft 152 comprises three functional spaces 158, 162, 164 that extend in an inner jacket 160 of the shaft 110. The functional spaces 158, 162, 164 serve as feedthrough channels for different purposes.

A camera 184 with suitable optics is attached distally of the distal shaft end 130. It comprises electronic components such as an image converter and an amplifier that create waste heat. The camera 184 is installed in heat-conducting fashion into a heat-conducting distal piece 186 of the shaft 110. The heat-conducting distal piece 186 is attached in heat-conducting fashion to the distal shaft end 130.

Furthermore, a light source 188 such as one or more LEDs is attached distally of the distal shaft end 130. The light source 188 also creates waste heat that can be dissipated via the distal piece 186 and the shaft 110.

A first functional space 158 serves to feed through lines 180, 182 that extend from a proximal shaft end 134 to a distal shaft end 130 of the shaft 110. In the present case, these lines 180, 182 are attached to the camera 184 and the light source 188 such that these can be controlled through the first functional space 158 and can be supplied with power.

A second functional space 162 serves to feed through an effector system 190, which is designed as a pair of coagulation forceps by way of example. The second functional space 162 is a working channel in particular. During operation, the effector system 190 also creates waste heat that can be dissipated via the shaft 110.

In the event of such an arrangement, the use of the described shaft 110 is particularly expedient since much waste heat that can be dissipated efficiently arises at a distal side in this case.

A third functional space 164 is designed as a fluid channel in the present case. The latter may merge into a fluid tube on a proximal side and/or be attached to a fluid line. By way of example, the third functional space 164 may serve to guide rinsing fluid, gases or other fluids.

A proximal portion has not been depicted in FIG. 7. It is understood that, for instance, a handle and/or suitable supply units and/or control units may be arranged there.

1-15. (canceled)

16. An endoscopic device comprising a shaft, wherein the shaft comprises:
a distal shaft end and a proximal shaft end opposite the distal shaft end,
an outer jacket that extends from the distal shaft end to the proximal shaft end,
an internal shaft portion that is at least partially enclosed circumferentially by the outer jacket and is arranged between the distal and the proximal shaft end,
at least one inner jacket that extends at least partially in the internal shaft portion and that defines at least one functional space; and
a sealed shaft interspace which is arranged in the internal shaft portion and outside of the functional space and in which a heat transfer unit is arranged, the latter filling at least a majority of the shaft interspace and being configured to dissipate heat from the distal shaft end.

17. The endoscopic device of claim 1, wherein the sealed shaft interspace with the heat transfer unit arranged therein forms a heat pipe.

18. The endoscopic device of claim 1, wherein the heat transfer unit comprises a gas exchange volume and a fluid transport layer, and the heat transfer unit comprises at least one heat transport means that is based on gas exchange, liquid transport and enthalpy of vaporization of the heat transport means.

19. The endoscopic device of claim 18, wherein the fluid transport layer is a porous element.

20. The endoscopic device of claim 18, wherein the fluid transport layer is arranged on an inner face of the outer jacket, and the fluid transport layer and the outer jacket are formed integrally.

21. The endoscopic device of claim 18, wherein the fluid transport layer is arranged on an outer face of the inner jacket, and the fluid transport layer and the inner jacket are formed integrally.

22. The endoscopic device of claim 18, wherein at least 90% of a cross-sectional area of the shaft interspace between the outer jacket and the at least one inner jacket are filled with the fluid transport layer and the heat transport means.

23. The endoscopic device of claim 20, wherein at least 90% of a cross-sectional area of the shaft interspace between the outer jacket and the at least one inner jacket are filled with the fluid transport layer and the heat transport means.

24. The endoscopic device of claim 16, further comprising at least one of a proximal and a distal thermal connection piece for heat emission and heat absorption, respectively.

25. The endoscopic device of claim 24, wherein the at least one thermal connection piece comprises a surface-increasing means for improving the heat emission or heat absorption, wherein the surface-increasing means is formed within the sealed region of the heat transfer unit and projects into the latter or is formed outside of the sealed region of the heat transfer unit and points away from the sealed region of the heat transfer unit.

26. The endoscopic device of claim 25, wherein the surface-increasing means is produced from a thermally conductive material produced from at least one of copper, copper alloys, silver and aluminum.

27. The endoscopic device of claim 16, wherein the outer jacket or the inner jacket comprises a thermally conductive element, the thermal conductivity of which is higher than that of the material of the outer jacket or of the inner jacket.

28. The endoscopic device of claim 24, further comprising a functional unit that is arranged at the distal shaft end, creates waste heat, and is configured to be thermally coupled to the heat transfer unit such that heat from the functional unit that creates waste heat is transferred via the distal thermal connection piece to the heat transfer unit.

29. The endoscopic device of claim 26, further comprising a functional unit that is arranged at the distal shaft end, creates waste heat, and is configured to be thermally coupled to the heat transfer unit such that heat from the functional unit that creates waste heat is transferred via the distal thermal connection piece to the heat transfer unit.

30. The endoscopic device of claim 27, further comprising a functional unit that is arranged at the distal shaft end, creates waste heat, and is configured to be thermally coupled to the heat transfer unit such that heat from the functional unit that creates waste heat is transferred via the distal thermal connection piece to the heat transfer unit.

31. The endoscopic device of claim 28, wherein the functional unit that creates waste heat comprises an electrical functional unit which by preference comprises at least one sensor or transmitter or receiver or at least one illumination unit or at least one effector unit or at least one camera unit.

32. The endoscopic device of claim 16, wherein the at least one functional space is configured to receive at least one functional unit that protrudes at least partially into the internal shaft portion or passes through the internal shaft portion, and wherein the functional unit protrudes beyond the sealed shaft interspace (distally or proximally.

33. The endoscopic device as claimed in claim 32, wherein all functional units that are guided through the internal shaft portion or are received therein are arranged in the at least one functional space and sealed vis-á-vis the shaft interspace by means of the inner jacket.

34. The endoscopic device of claim 16, further comprising a handle that is coupled or couplable to the proximal shaft end or a region adjacent to the proximal shaft end.

35. The endoscopic device of claim 16, wherein the endoscopic device comprises an endoscope.
